(19) United States
(12) Plant Patent Application Publication
(10) Pub. No.: US 2024/0298556 P1
(43) Pub. Date: Sep. 5, 2024

(54) STRAWBERRY PLANT NAMED 'HONGHUI'

(50) ;

(71) Applicant: Zordi, Inc., Boston, MA (US)

(72) Inventor: Iyeoung Choi, Hongseong (KR)

(73) Assignee: Zordi, Inc., Boston, MA (US)

(21) Appl. No.: 18/176,716

(22) Filed: Mar. 1, 2023

(51) Int. Cl. A01H 6/74

(52) U.S. Cl. CPC A01H 6/7409
USPC PLT/208

(57) ABSTRACT

A new and distinct variety of strawberry plant named ‘HongHui’, particularly selected for its very large fruit size. strong fruit glossiness, aroma, juiciness, and very early and consistent flowering, is disclosed.

Latin Name: Botanical classification: Fragaria x ananassa.

Varietal denomination:

The varietal denomination of the claimed variety of strawberry plant is ‘HongHui’.

### BACKGROUND OF THE INVENTION

Cultivated strawberry is a hybrid species of the genus Fragaria that is grown worldwide for its fruit. Modern strawberry was first bred in Brittany, France, in the 18^{th} century by crossing Fragaria virginiana with Fragaria chiloensis. Strawberry fruit is an aggregate accessory fruit, with the fleshy part of the fruit being derived from the receptacle that holds the ovaries.

Strawberry varieties vary widely in color, size, shape, flavor, season of ripening, degree of fertility, and susceptibility to disease. Certain varieties vary in foliage, and some vary in the relative development of their reproductive organs. Typically, strawberry flowers appear hermaphroditic in structure, but function as either male or female. Generally, commercial production of strawberry plants involves propagation from runners and distribution as either plugs or bare root plants. Cultivation is either perennial or annual plasticulture. During the off season, strawberries can also be produced in greenhouses.

Strawberry fruit is widely appreciated for its characteristic bright red color, aroma, juicy texture, and sweetness. Strawberry fruit is a popular fruit that is generally consumed either fresh or in prepared foods, such as preserves and baked goods.

Strawberry is an important and valuable fruit crop. Accordingly, there is a need for new varieties of strawberry plants. In particular, there is a need for improved varieties of strawberry plant that are stable, high yielding, and agronomically sound.

### SUMMARY OF THE INVENTION

In order to meet these needs, the present invention is directed to an improved variety of strawberry plant. In particular, the invention relates to a new and distinct variety of strawberry plant (Fragaria x ananassa), which has been denominated as ‘HongHui’.

Strawberry plant variety ‘HongHui’ originated from a cross made in 2016 between the proprietary female parent ‘H2’ (itself a hybrid between peach-flavored ‘Tokun’ (South Korean Plant Variety Protection Certificate No. 2013-9; Japanese Plant Variety Protection Registration No. 21165) and ‘Sulhyang’ (South Korean Plant Variety Protection Certificate No. 2012-427)) and the male parent variety ‘Kingsberry’ (South Korean Plant Variety Protection Application No. 2016-319). In 2017, 250 progeny plants from this initial cross were propagated and selected for excellent fruit quality and unique scent. Selected plants were propagated via stolons and underwent further testing for two years (2018-2019). After a cultivation test in Hongseong, South Korea in 2019, variety ‘HongHui’ was specifically identified and selected. ‘HongHui’ was subsequently asexually propagated via stolon cuttings, and underwent further testing in Hongseong, South Korea for four months (December 2019-March 2020). The present variety has been found to be stable and reproduce true to type through successive propagations via stolon cuttings.

‘HongHui’ was selected for its very large fruit size, strong fruit glossiness, aroma, juiciness, and its very early and consistent flowering. The early timing of its consistent flowering and its excellent, vigorous growth enables high yield early in the growing season.

### DESCRIPTION OF THE DRAWINGS

This new strawberry plant is illustrated by the accompanying photographs. The colors shown are as true as can be reasonably obtained by conventional photographic procedures. Unless otherwise indicated, the photographs are of plants that are 6-8 months old.

FIGS. 1A-1C illustrate a series of developing fruit of the variety ‘HongHui’. FIG. 1A shows four whole, immature, green fruit of the variety at multiple developmental stages. FIG. 1B shows two whole, immature fruit of the variety, including one fruit that is beginning to redden (right). FIG. 1C shows three whole, immature fruit of the variety; this includes one green fruit (left), one fruit beginning to redden (behind), and one fruit almost fully reddened (right).

FIG. 2 illustrates a whole mature fruit of the variety ‘HongHui’, displaying its high glossiness and the outward attitude of the sepals.

FIG. 3 illustrates longitudinal fruit sections of a mature fruit of the variety ‘HongHui’ and a measurement of the maximum fruit length (86.32 mm).

FIG. 4 illustrates a transversal measurement of a longitudinal fruit section of a mature fruit of the variety ‘HongHui’ and a measurement of the maximum fruit diameter (46.87 mm).

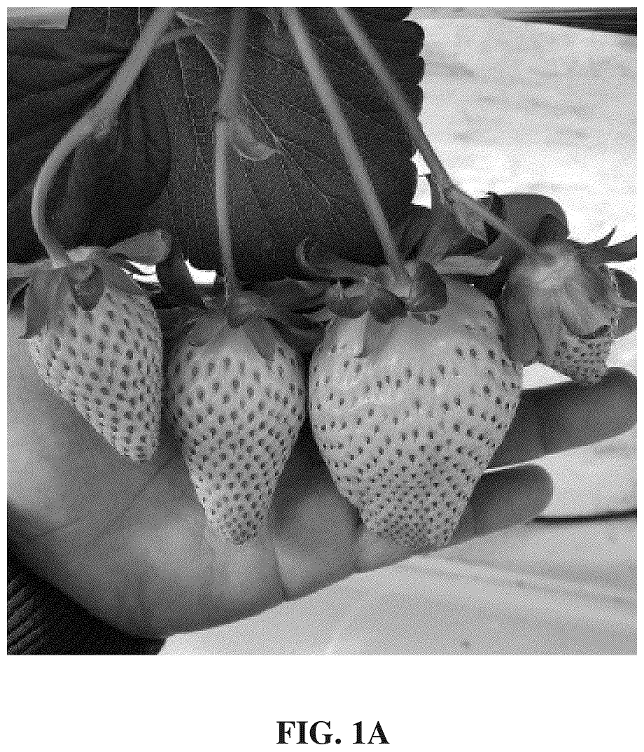

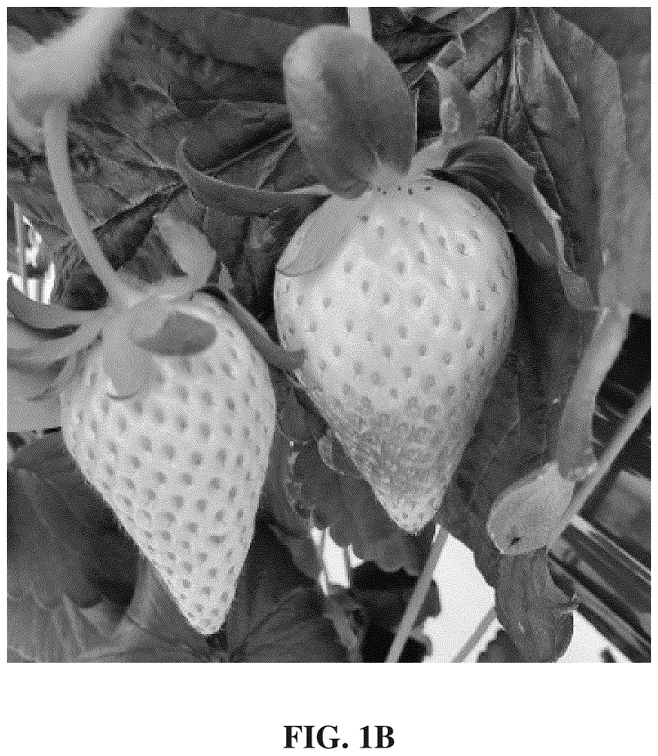

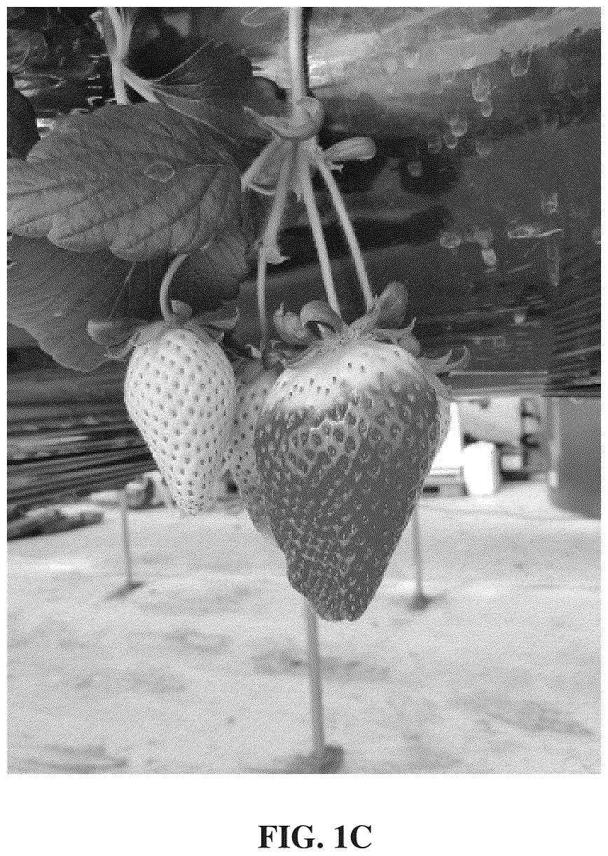

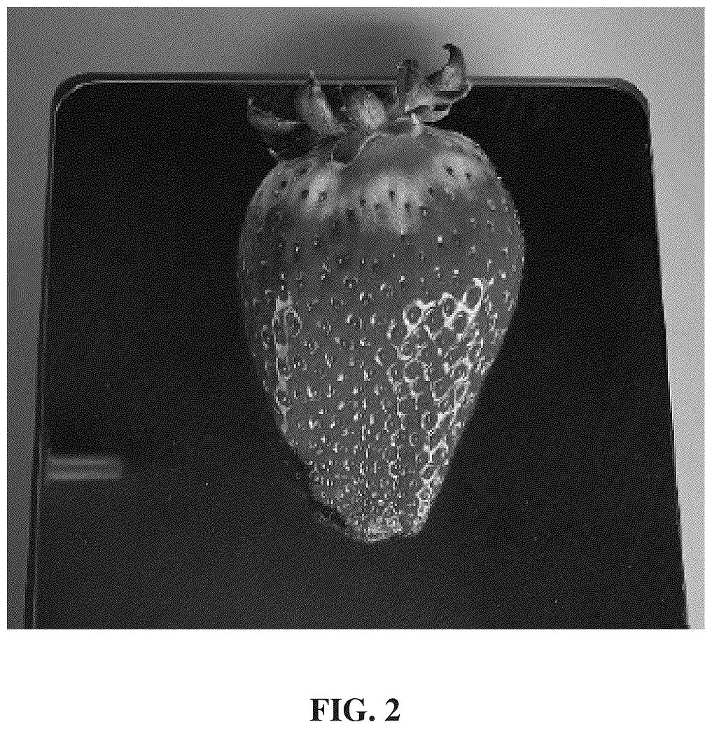

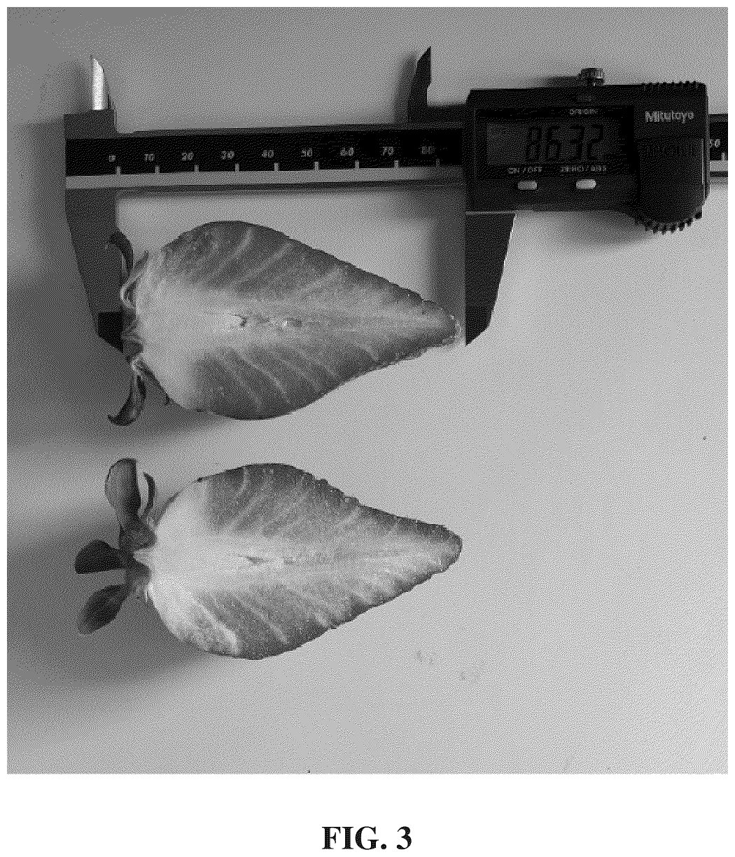

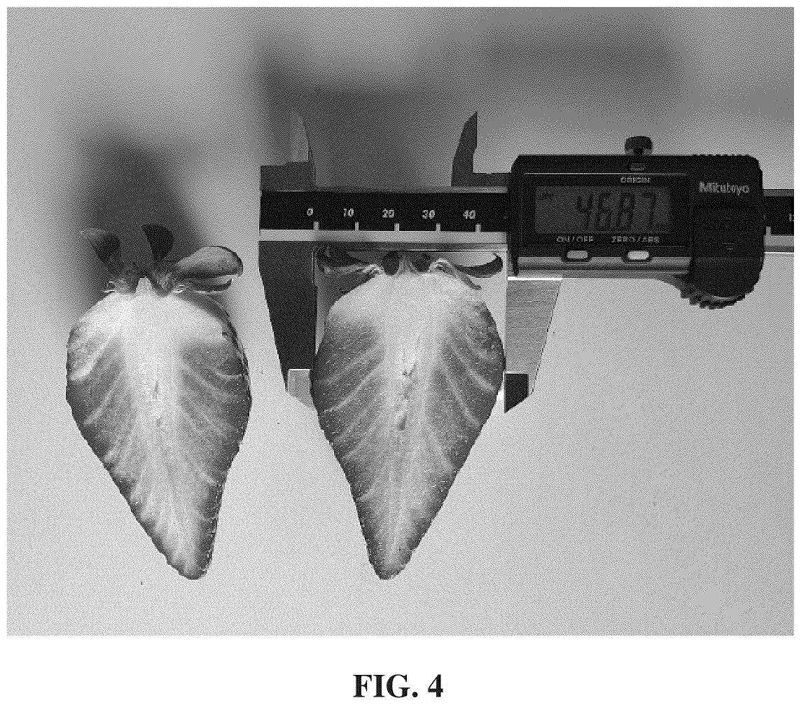

### DETAILED BOTANICAL DESCRIPTION

The following detailed description sets forth the distinctive characteristics of ‘HongHui’. The data which define these characteristics is based on observations taken in Hongseong, South Korea from December 2019 to March 2020. This description is in accordance with UPOV terminology. Color designations, color descriptions, and other phenotypical descriptions may deviate from the stated values and descriptions depending upon variation in environmental, seasonal, climatic, and cultural conditions. ‘HongHui’ has not been observed under all possible environmental conditions. The botanical description of ‘HongHui’ was taken from plants that were 6-8 months old. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.
Classification:
   Genus/species.—Fragaria x ananassa.
   Common name.—Strawberry.
   Denomination.—‘HongHui’.
Parentage:
   Female parent.—‘H2’ (unpatented).
   Male parent.—‘Kingsberry’ (South Korean Plant Variety Protection Application No. 2016-319).
Plant:
   Number of stolons per plant.—25.
   Growth habit.—Upright to semi-upright.
   Density of foliage.—Medium.
   Vigor.—Strong.
   Measured height.—35.7 cm.
   Width/Spread.—31 cm.
   Type.—Short day.
   U.S.D.A. Hardiness Zone.—7a-10b (best for 9a).
   Fusarium wilt resistance.—Strong.
   Anthracnose resistance.—Strong.
   Root color.—White.
   Time to initiate roots.—3 days.
   Time to produce a rooted plant.—10 days.
Stolon:
   Anthocyanin coloration.—Weak.
   Anthocyanin color.—Green.
Leaf:
   Size.—Very large.
   Length.—21 cm.
   Width.—19 cm.
   Division.—Compound; 3 leaflets.
   Blistering.—Medium.
   Glossiness.—Medium.
   Terminal leaflet.—Shape: Obtuse. Length: 21 cm. Width: 19 cm. Length/width ratio: Medium. Shape of apex: Serrate to crenate. Shape of base: Acute. Margin: Serrate to crenate. Margin incision depth: Shallow. Shape in cross section: Straight. Color of leaf upper surface: Medium green. Texture of upper surface: Rough. Texture of lower surface: Soft.
   Petiole.—Leaflets: Long. Length: Long; 12 cm. Attitude of hairs: Upwards.
   Stipule.—Anthocyanin coloration: Medium.
Inflorescence:
   Average number of flowers per peduncle.—11.8.
   Position of inflorescence in relation to foliage.—Clearly beneath.
   Length.—21 cm.
   Peduncle.—Length: 5 cm.
   Flower.—Flower diameter: Medium to large. Arrangement of petals: Touching. Size of calyx in relation to corolla: Larger. Stamen: Present.
   Sepal.—Number: 5-7. Length: 2.5 cm. Width: 2.1 cm. Shape: Outward.
   Petal.—Number: 5-6. Length: 2.1 cm. Width: 1.9 cm. Length/width ratio: High. Color of upper surface: White. Overall shape: Elliptic.
   Flowering.—

Flowering runners: Present. Flowering season: Winter.
Fruit:
   Fruit size.—Overall size: Very large. Average fruit weight: 25 g. Average fruit length: 54 mm. Average fruit diameter: 36 mm. Maximum fruit weight: 75 g. Maximum fruit length: 86.32 mm. Maximum fruit diameter: 46.87 mm. Position of maximum diameter: Moderately towards the calyx.
   Shape.—Conical.
   Shape of apex.—Acute.
   Shape at calyx end.—Rounded.
   Significant difference between primary and secondary fruit shape.—Absent.
   Fruit color.—RHS 34A (Vivid Reddish Orange).
   Glossiness.—Strong.
   Width of band without achenes.—Very narrow to narrow.
   Position of achenes.—Level with surface.
   Position of calyx attachment.—Level with fruit.
   Attitude of sepals.—Outwards.
   Diameter of calyx in relation to diameter of fruit.—Slightly larger.
   Firmness.—Firm (232 g·mm⁻²).
   Color of flesh (excluding core).—Medium red.
   Color of core.—Light red.
   Average sweetness.—11.5 Brix.
   Achenes.—Number per berry: 150-200. Color of upper (sunward) side: Yellow.
   Fruiting.—Time of beginning of fruit ripening: November. Fruiting season: 6 months. Marketable yield per season: 800 g per plant per season.

### COMPARISON WITH PARENTAL AND REFERENCE VARIETIES

‘HongHui’ differs from the female parent ‘H2’ (crossbreed of ‘Tokun’ (South Korean Plant Variety Protection Application No. 2013-9; Japanese Plant Variety Protection Registration No. 21165) and ‘Sulhyang’ (South Korean Plant Variety Protection Application No. 2012-427)) in that ‘HongHui’ pollinates well and produces larger fruit than ‘H2’. Moreover, the leaves of variety ‘HongHui’ are larger than the leaves of ‘H2’. In addition, ‘HongHui’ produces fruit with a medium red color, while ‘H2’ produces fruit with a light pink color. Further, ‘HongHui’ plants have less resistance to powdery mildew than ‘H2’ plants.

‘HongHui’ differs from the male parent ‘Kingsberry’ (South Korean Plant Variety Protection Application No. 2016-319) in that the fruits produced by ‘HongHui’ are more firm and have higher acidity than the fruits produced by ‘Kingsberry’. Moreover, ‘HongHui’ produces fruit with a larger maximum diameter than the fruits of ‘Kingsberry’. In addition, ‘HongHui’ plants are more vigorous than ‘Kingsberry’ plants. Further, the leaves of ‘HongHui’ are larger than the leaves of ‘Kingsberry’. While the first peduncle (flush) of ‘HongHui’ happens around the same time as the first peduncle of ‘Kingsberry’, the second peduncle of ‘HongHui’ comes out faster than the second peduncle of ‘Kingsberry’. Additionally, the ‘HongHui’ plants have higher resistance to anthracnose, Fusarium wilt (Fusarium oxysporum), and Phytophthora blight than ‘Kingsberry’ plants.

‘HongHui’ differs from the commercial variety ‘Sulhyang’ (South Korean Plant Varity Protection Certificate No. 2012-427) in that ‘HongHui’ produces very large fruit, while ‘Sulhyang’ produces large fruit. Additionally, the fruit of ‘HongHui’ has strong glossiness, while the fruit of ‘Sulhyang’ has medium glossiness. Moreover, ‘HongHui’ plants have strong vigor, while ‘Sulhyang’ plants have medium vigor. Further, ‘HongHui’ produces larger flowers than ‘Sulhyang’. In addition, ‘HongHui’ plants produce flower trusses consistently at a faster rate than ‘Sulhyang’ plants. Furthermore, the leaves of ‘HongHui’ are very large, while the leaves of ‘Sulhyang’ are medium in size. Moreover, the fruit of ‘HongHui’ has a small or absent hollow center, while the fruit of ‘Sulhyang’ has a medium hollow center. Further, the time of fruit maturation is very slow for ‘HongHui’, while the time of fruit maturation for ‘Sulhyang’ is fast.

We claim:

1. A new and distinct variety of strawberry plant named ‘HongHui’ as shown and described herein.
